(19) United States
(12) Patent Application Publication
(10) Pub. No.: US 2015/0258202 A1
(43) Pub. Date: Sep. 17, 2015

(54) ANTHOCYANIDIN COMPLEX FOR THE TREATMENT OF MULTIPLE MYELOMA

(71) Applicant: SAPIOTEC GMBH, Würzburg (DE)

(72) Inventors: Norbert Roewer, Wurzbrug (DE); Jens Broscheit, Wurzburg (DE)

(21) Appl. No.: 14/432,654

(22) PCT Filed: Oct. 17, 2013
(86) PCT No.: PCT/EP2013/071779

§ 371 Date: Mar. 31, 2015

(30) Foreign Application Priority Data
Oct. 17, 2012 (EP) 12188789.7

(51) Int. Cl. A61K 47/48; A61K 31/724; A61K 45/06

(52) U.S. Cl. CPC A61K 47/48061; A61K 45/06; A61K 31/724

(57) ABSTRACT

The subject matter of the invention is a complex of delphinidin and a sulfoalkyl ether β-cyclodextrin for use as a medicinal drug, in particular in the treatment of multiple myeloma.

The invention relates to a complex of an anthocyanidin and a sulfoalkyl ether β-cyclodextrin and also compositions comprising anthocyanidin or salts thereof as a medicament for the treatment of cancer.

Anthocyanidins are zymochromic pigments having antioxidative properties, which occur in most higher terrestrial plants. Anthocyanidins are sugar-free (aglycones) and closely related to the sugar-containing anthocyans (glycosides), both of which fall under the generic heading of anthocyans.

Multiple myeloma is a degeneration of plasma cells. Plasma cells are cells of the immune system which produce antibodies for the battle against diseases and infections. These cells are transported by the bloodstream, inter alia, into the bone marrow where they accumulate and cause permanent damage to the healthy tissue for which notable symptoms are bone fractures, increased calcium levels (hypercalcemia) or even renal failure. The origin of the bone damage lies in the rapid proliferation of the myeloma cells and liberation of the osteoclast activator Il-6, which activates the osteoclasts responsible for bone substance resorption, which, as a result, leads to damage of the bone substance and thus to bone fractures. Since the myeloma cells in the bone marrow displace the normal cells, the production of normal blood cells is also affected, particularly the white and red blood cells too, which firstly increases the risk of infection and secondly can lead to anemia. The decreasing number of blood platelets also leads to deterioration in blood clotting. The average life expectancy is poor in affected patients at 6 months following diagnosis of the disease even if it may be extended by a few years by high-dose chemotherapy and autologous stem cell transplantation. Therefore, there is an acute need for alternative and effective remedies and methods of treatment.

The object of the present invention is to provide an effective medicament for the treatment of multiple myeloma.

This object is achieved by a complex of an anthocyanidin and a sulfoalkyl ether β-cyclodextrin according to claims 1-2. Advantageous embodiments of the invention are disclosed in the subclaims.

Some terms used in the context of the invention will first be explained.

The complex according to the invention or the composition according to the invention is used for the treatment of a subject or individual suffering from multiple myeloma. The term “subject” includes living animals and humans. The term “composition comprising at least one anthocyanidin” includes an anthocyanidin as such without further components. The purpose of this treatment is the at least partial killing or neutralization of the myeloma cells. “Neutralization” or “killing” signifies, in the context of the present invention, the at least partial destruction or disintegration or inactivation or prevention of myeloma cell proliferation. “Multiple myeloma” is a cancer of plasma cells. The stages of multiple myeloma may be identified by means of the International Staging System (ISS). The ISS is based on the assessment of blood test results relating to β₂-microglobulin (β₂-M) and albumin, where the two in combination with each other allow the most reliable prognosis for multiple myeloma compared to other test factors. The criteria for diagnosing the different stages corresponding to the ISS for myeloma are, for stage I: β₂-M<3.5 mg/dL and albumin≧3.5 g/dL, for stage II: β₂-M<3.5 mg/dL or β₂-M 3.5-5.5 mg/dL and albumin<3.5 g/dL and for stage III: β₂-M>5.5 mg/dL. The stages of multiple myeloma are normally classified in one of the various myeloma categories. Multiple myeloma can be asymptomatic or symptomatic. In asymptomatic myeloma patients, no impairments or symptoms of the organs and tissues are apparent. Impairments of the organs or tissues caused by myeloma include hypercalcemia, impaired kidney function, anemia and bone injuries. Asymptomatic myeloma includes smoldering multiple myeloma (SMM) and stage I multiple myeloma. SMM is characterized by monoclonal protein and a slight increase in plasma cells in the bone marrow. Indolent multiple myeloma (IMM) is characterized by low amounts of monoclonal protein and a raised number of plasma cells in the bone marrow. Patients with multiple myeloma are also characterized by their disease status. The disease status is determined based on whether the patient has already received therapy and, if so, with what result. Patients with renewed or repeated diagnosis of the disease, in the context of the present invention, are individuals who are suffering from myeloma and have already been treated. Patients who have already received therapy fall into various classes mentioned as follows. Responsive disease: refers to myeloma which responds to therapy such that the M-protein level decreases by at least 50%; stable disease: refers to myeloma which does not respond to treatment (i.e. no reduction of the M-protein level by 50% is achieved), but does not progress further, i.e. no deterioration occurs; progressive disease: refers to active myeloma which deteriorates, i.e. an increase in the M-protein level and more pronounced impairments of the organs and tissues. In the majority of cases, the relapsed disease and/or refractory disease mentioned below may also be classified as progressive disease. Relapsed disease: refers to myeloma which initially responds to therapy but thereafter reverts to the progression stage. Refractory disease: refers both to myeloma which does not respond to first-line therapy and to relapsed myeloma which no longer responds to subsequent treatments. The latter may also be referred to as a relapsed disease.

The present invention also relates to a method for the treatment of a subject suffering from multiple myeloma, wherein the subject is administered a therapeutically effective amount of the complex according to the invention or the composition according to the invention. Multiple myeloma may be treated in all of the stages, categories or disease statuses described above. The complex according to the invention or the composition according to the invention may be administered alone or in combination with at least one other therapeutic agent for reducing one or more symptoms of multiple myeloma. The complex according to the invention or the composition according to the invention may be administered simultaneously with the other therapeutic agent, which may be a constituent of the same composition or is provided in another composition. Alternatively, the complex according to the invention or the composition according to the invention may be administered before or after the administration of the other therapeutic agent. The complex according to the invention or the composition according to the invention may be administered by the same or another route of administration as the other therapeutic agent. The therapeutic agents may be chemotherapeutic agents, supportive therapeutic agents or a combination thereof. “Chemotherapeutic agent” is an agent which is toxic to cancer cells. Examples of chemotherapeutic agents which may be used in the context of the present invention include bortezomib (Velcade®, Millennium), melphalan, prednisone, vincristine, carmustine, cyclophosphamide, dexamethasone, thalidomide, doxorubicin, cisplatin, etoposide and cytarabine. In a particularly preferred embodiment of the invention, the complex according to the invention or the composition according to the invention is used in combination with bortezomib (Velcade®). In a further preferred embodiment of the invention, the complex according to the invention or the composition according to the invention is used in combination with melphalan. A “supportive therapeutic agent” is an agent which is used to reduce the symptoms and complications of multiple myeloma. Examples of supportive therapeutic agents are bisphosphonates, growth factors, antibiotics, diuretics and analgesics.

Examples of antibiotics include sulfur-containing drugs, penicillins (e.g. benzylpenicillin, p-hydroxy-benzylpenicillin, 2-pentenylpenicillin, N-heptyl-penicillin, phenoxymethylpenicillin, phenethicillin, methicillin, oxacillin, cloxacillin, dicloxacillin, flucloxacillin, nafcillin, ampicillin, amoxicillin, cyclacillin, carbenicillin, ticarcillin, piperacillin, azlocillin, mezlocillin, mecillinam, amdinocillin), cephalosporin and derivatives thereof (e.g. cephalothin, cephapirin, cephacetrile, cephazolin, caphalexin, cephandine, cefadroxil, cefamandol, cefuroxime, ceforanide, cefoxitin, cefotetan, cefaclor, cefotaxime, ceftizoxime, ceftrioxone, ceftazidime, moxalactam, cefoperazone, cefixime, ceftibuten and cefprozil), oxolinic acid, amifloxacin, temafloxacin, nalidixic acid, piromidic acid, ciprofloxacin, cinoxacin, norfloxacin, perfloxacin, rosaxacin, ofloxacin, enoxacin, pipemidic acid, sulbactam, clavulinic acid, β-bromopenicillanic acid, β-chloro-penicillanic acid, 6-acetylmethylenepenicillanic acid, cephoxazole, sultampicillin, formaldehyde hydate esters of adinocillin and sulbactam, tazobactam, aztreonam, sulfazethin, isosulfazethin, norcardicins, m-carboxyphenyl phenylacetamidomethylphosphonate, chlortetracycline, oxytetracyline, tetracycline, demeclocycline, doxycycline, methacycline and minocycline. Examples of bisphosphonates include etidronate (Didronel), pamidronate (Aredia), alendronate (Fosamax), risedronate (Actonel), zoledronate (Zometa), ibandronate (Boniva). Examples of diuretics include thiazide derivatives such as amiloride, chlorothiazide, hydrochlorothiazide, methylchlorothiazide and chlorthalidone. Examples of growth factors include granulocyte colony-stimulating factor (G-CSF), granulocyte-macrophage colony-stimulating factor (GM-CSF), macrophage colony-stimulating factor (M-CSF), multi-colony-stimulating factor, erythropoietin, thrombopoietin, oncostatin M and interleukins. Examples of analgesics include opiates (e.g. morphine), COX-2 inhibitors (e.g. rofecoxib, valdecoxib and celecoxib), salicylates (e.g. ASPIRIN, choline magnesium trisalicylate, salsalate, dirunisal and sodium salicylate), propionic acid derivatives (e.g. fenoprofen calcium, ibuprofen, ketoprofen, naproxen and naproxen sodium), indoleacetic acid derivatives (e.g. indomethacin, sulfindac, etodalac and tolmetin), fenamates (e.g. mefenamic acid and meclofenamate), benzothiazine derivatives or oxicams (e.g. mobic or piroxicam) or pyrrolactic acid (e.g. ketorolac).

The term “treatment” signifies, in the context of the present invention, complete or partial achievement of the following specified results: completely or partially reducing the clinical picture; improving at least one of the clinical symptoms or indicators associated with the disease; delaying, suppressing or providing protection from the progression of the disease; or completely or partially delaying, suppressing or providing protection from onset or development of the disease. The subject to be treated is a human or animal, preferably a mammal. Veterinary medical treatment, besides the treatment of livestock or wild animals (e.g. sheep, cats, horses, cows, pigs), also includes laboratory animals (e.g. rats, mice, guinea pigs, monkeys).

In one embodiment of the invention, the subject treated with the complex according to the invention or the composition according to the invention and optionally further therapeutic agents, is subjected to radiation therapy and/or is prepared for stem cell therapy. The complex according to the invention or the composition according to the invention can be used, preferably in combination with optional further therapeutic agents, in the course of induction therapy to reduce the tumor burden in advance of stem cell transplantation, but also in the course of stem cell transplantation and/or after stem cell transplantation.

The complex according to the invention or the composition according to the invention is preferably provided and administered as a pharmaceutical composition. The term “pharmaceutical composition” includes one or more active ingredients and one or more inert substances which function as carriers for the active ingredient or active ingredients. The pharmaceutical compositions allow the complex according to the invention or the composition according to the invention to be administered by the oral, parenteral, including subcutaneous, intramuscular and intravenous, ophthalmical, pulmonary or nasal route. A parenteral administration form may be, for example, a solution, suspension or dispersion. An ophthalmic, pulmonary or nasal administration form may be, for example, an aerosol, solution, suspension or dispersion. Appropriate techniques for the formulation and administration are known from the prior art; for example, see “Remington's Pharmaceutical Sciences” (Mack Publishing Co., Easton Pa.). For example, the compositions and complexes according to the invention may be administered to a subject intravenously by means of a pharmaceutically acceptable carrier (e.g. physiological saline solution). A formulation in aqueous solution, preferably in physiologically acceptable buffers (e.g. Hank's solution, Ringer's solution or physiologically buffered saline solution), is suitable for injection. For parenteral administration, including intravenous, subcutaneous, intramuscular and intraperitoneal administration, an aqueous or oily solution or a solid formulation is also useful. The proportion of active ingredient in the pharmaceutical composition may vary and is typically between 2 and 60% by weight of the dose unit. The proportion of active ingredient is accordingly selected such that an effective dose is achieved.

“Salt” or “pharmaceutically acceptable salt” is any salt of a compound of the present invention, acceptable from a pharmaceutical standpoint, which can liberate the pharmaceutically effective active ingredient or active metabolite thereof after administration. Salts of the compositions and complexes of the present invention may be derived from inorganic or organic acids and bases.

The anthocyanidin may be used in “pure form” or “purified”, which signifies that undesired components have been removed.

“Anthocyanidins” have the basic structure shown below:

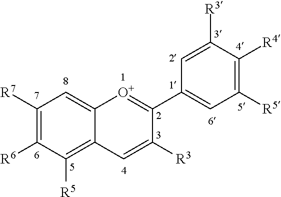

The substituents in this formula are selected from the group consisting of hydrogen, hydroxyl group, and methoxy group.

Cyclodextrins, which can be complexed with the anthocyanidin in accordance with the invention, are cyclic oligosaccharides of glucose molecules linked by α-1,4-glycosidic bonds. β-cyclodextrin has seven glucose units. In a sulfoalkyl ether β-cyclodextrin, hydroxyl groups of the glucose unit in a sulfoalkyl alcohol are etherified. According to the invention, generally only some of the 21 hydroxyl groups of a β-cyclodextrin are etherified. The preparation of sulfoalkyl ether cyclodextrins is familiar to those skilled in the art and is described, for example, in U.S. Pat. No. 5,134,127 or WO 2009/134347 A2.

Sulfoalkyl ether groups are used in cyclodextrins in the prior art to increase their hydrophilicity or water solubility. Sulfoalkyl ether groups contribute to a particular degree to increasing the stability of the complex of anthocyanidins and correspondingly substituted β-cyclodextrin and thus substantially improve the storage stability and formulatability of the anthocyanidins, which are particularly sensitive to oxidation. The complex according to the invention may be formulated as an aqueous solution or solid, stable on storage, as will be shown in even more detail below.

In accordance with the invention, particular preference is given to complexing with a sulfobutyl ether β-cyclodextrin (SEB-β-CD). A possible explanation for this, which does not limit the scope of protection, is that the negatively charged sulfobutyl units interact electrostatically with the positively charged anthocyanidins and, in terms of the alkyl groups, the butyl group has the optimal length to enable an appropriate steric interaction.

The degree of substitution of the cyclodextrin with sulfoalkyl ether groups is preferably 3 to 8, more preferably 4 to 8, more preferably 5 to 8, more preferably 6 to 7. Suitable sulfobutyl ether β-cyclodextrins having a mean degree of substitution of 6 to 7 are described, for example, in the cited WO 2009/134347 A2 and are commercially available under the trade name Captisol®. Corresponding cyclodextrins having a degree of substitution of 4 to 5, for example 4.2, can likewise be used.

The anthocyanidins used in pure, salt or complexed form in accordance with the invention are preferably selected from the group consisting of aurantinidin, cyanadin, delphinidin, europinidin, luteolinidin, pelargonidin, malvidin, peonidin, petunidin and rosinidin. The chemical structure corresponds to Formula I given above with the following substitution pattern

| | R^{3′} | R^{4′} | R^{5′} | R³ | R⁵ | R⁶ | R⁷ |
|---|---|---|---|---|---|---|---|
| Aurantinidin | —H | —OH | —H | —OH | —OH | —OH | —OH |
| Cyanidin | —OH | —OH | —H | —OH | —OH | —H | —OH |
| Delphinidin | —OH | —OH | —OH | —OH | —OH | —H | —OH |
| Europinidin | —OCH₃ | —OH | —OH | —OH | —OCH₃ | —H | —OH |
| Luteolinidin | —OH | —OH | —H | —OH | —OH | —H | —OH |
| Pelargonidin | —H | —OH | —H | —OH | —OH | —H | —OH |
| Malvidin | —OCH₃ | —OH | —OCH₃ | —OH | —OH | —H | —OH |
| Peonidin | —OCH₃ | —OH | —H | —OH | —OH | —H | —OH |
| Petunidin | —OH | —OH | —OCH₃ | —OH | —OH | —H | —OH |
| Rosinidin | —OCH₃ | —OH | —H | —OH | —OH | —H | —OCH₃ |

Particular preference is given to delphinidin in the context of the invention.

The invention also relates to an aqueous solution of the composition according to the invention or the complex according to the invention for use as a medicament, particularly for the treatment of multiple myeloma.

The preparation of the complex according to the invention, and also a corresponding aqueous solution comprises the following steps:
a) preparing an aqueous solution of the sulfoalkyl ether β-cyclodextrin,
b) adding the anthocyanidin and mixing to prepare the complex.

In step a), preference is given to preparing an aqueous solution comprising 5 to 10% by weight of the cyclodextrin used. It is particularly preferred in the context of the invention, if the pH of the aqueous solution is adjusted during or after, but preferably before, addition of the anthocyanidin, preferably delphinidin, to a pH of 7 or less, preferably 6 or less, more preferably 5 or less, more preferably 4 to 5. It has been shown that, at this pH, a higher concentration of the complex in aqueous solution can be established.

The concentration of the anthocyanidin, calculated as the chloride, is preferably at least 0.5 mg/ml, more preferably at least 1.0 mg/ml, more preferably at least 1.5 mg/ml, more preferably 2.0 mg/ml. In the context of a preferred embodiment, the particularly preferred concentration range of at least 2.0 mg/ml can be established in particular in an aqueous solution having a pH between 4 and 5.

In the context of the preparation, the mixing of the constituents of the aqueous solution can be accomplished by stirring with a preferred period for the mixing of 2 to 20 h. The mixing is preferably carried out in the dark in order to avoid light-induced oxidation.

The invention further relates to a solid for use as a medicament, particularly for the treatment of multiple myeloma, which can be obtained in accordance with the invention by removing the solvent from an aqueous solution according to the invention described above.

The removal can preferably be effected by freeze drying (lyophilization). Both the aqueous medicinal solution according to the invention and the medicinal solid have good storage stability.

The invention will now be described further in the examples which follow with reference to the attached figures without being restricted to them.

I. Preparation of a Complex of the Anthocyanidin Delphinidin and Cyclodextrins

1. Materials Used

The following cyclodextrins are used:

| | |
|---|---|
| α-CD | ID No: CYL-2322 |
| β-CD | ID No: CYL-3190 |
| γ-CD | ID No: CYL-2323 |
| (2-Hydroxypropyl)-β-CD | ID No: L-043/07 |
| Sulfobutyl ether β-CD | ID No: 47K010111 |

Delphinidin chloride was purchased from Extrasynthese.

2. Determination of the Delphinidin Content

A reversed-phase HPLC method was used to determine the delphinidin chloride content in the delphinidin-containing compositions. The following reagents were used for this purpose:

Purified water

Methanol for chromatography

Formic acid, p.a.

1 M hydrochloric acid as a volumetric solution.

The column used was a Waters X Bridge® C18, 35 μl, 150 mm×4.6 mm.

The mobile phases were as follows:
Phase A: Water 950 ml, methanol 50 ml, formic acid 10 ml
Phase B: Water 50 ml, methanol 950 ml, formic acid 10 ml

The following gradient program was used:

| Time [min] | Percent phase B |
|---|---|
| 0 | 0 |
| 5 | 0 |
| 25 | 60 |
| 30 | 100 |

Stop time: 35 min

After-run time (post time): 8 min

Flow rate: 1 ml/min

Injection volume: 20 μl

Column temperature: 30° C.+/−2° C.

UV/Vis detector: 530 μm for the assay, 275 μm for the detection of impurities

Integrator: area

Solutions and Sample Preparation:
Dilution solution 1: Mixture of 100 ml of methanol and 2.6 ml of 1 M HCl
Dilution solution 2: Mixture of 100 ml of 40% methanol and 2.6 ml of 1 M HCl

Calibration solution: A reference solution of delphinidin was prepared by weighing 10 mg of delphinidin chloride into a 10 ml flask and dissolving in dilution solution 1. After dissolution, the solution was diluted approximately 10-fold with dilution solution 2 to produce an approximate concentration of 0.1 mg/ml.

The control calibration solution was prepared in the same manner. The calibration solutions were immediately analyzed by HPLC since delphinidin chloride is unstable in solution.

Preparation of the Test Solutions:

To determine the delphinidin content of the solids prepared according to the invention (for preparation see below), approximately 50 mg of this composition were weighed into a 10 ml flask. This was then dissolved in dilution solution 2 and further diluted with the same dilution solution 2 until an approximate concentration of delphinidin of 0.1 mg/ml was established.

The determination of the delphinidin content in the samples was calculated with the aid of the Agilent ChemStation software using calibration with the external standard described.

### EXAMPLE 1

### Complexing of delphinidin with SBE-β-CD

In this example, the complexing of delphinidin by various cyclodextrins and the solubility of the complex in aqueous solution are investigated.

Neutral aqueous solutions were prepared comprising 10% by weight of the respective cyclodextrin. Due to the lack of solubility of β-CD, a concentration of only 2% by weight was selected.

Glass flasks were filled with 5 ml each of aqueous cyclodextrin solution and pure water. An excess of delphinidin chloride was then added. The required excess amount was 10 mg for the solutions of α-, β- and γ-cylcodextrin and 15 mg for the solutions of HPBCD (2-hydroxypropyl-β-cyclodextrin) and SBE-β-CD.

The suspensions were stirred at 30° C. for 20 h in the dark. The suspension was then filtered through a membrane filter of 0.22 μm pore size.

The solubilities achievable are shown in Table 1 below.

| | Cyclodextrin | Delphinidin |
|---|---|---|
| Cyclodextrin | concentration | chloride |
| — | 0 | 0.07 mg/ml |
| α-CD | 10% | 0.14 mg/ml |
| β-CD | 2% | 0.05 mg/ml |
| γ-CD | 10% | 0.21 mg/ml |
| HPBCD | 10% | 0.19 mg/ml |
| SBE-β-CD | 10% | 0.66 mg/ml |

It can be seen that the complexing and the increased solubility thereby effected is far better for SBE-β-CD than for the other cyclodextrins.

### EXAMPLE 2

### Influence of pH

In this example, the influence of the pH on the solubility of a delphinidin-SBE-β-CD in aqueous solution was investigated. Aqueous solutions of SEB-β-CD were prepared according to the procedure of Example 1, but these solutions were adjusted with 1 M HCl to the acid pH values given in Table 2. Delphinidin chloride was then added according to the procedure of Example 1 and further processed with the only exception that the stirring time was limited to 2.5 h. The results are shown in Table 2 below.

| | Delphinidin |
|---|---|
| pH | chloride |
| 6.0 | 0.60 mg/ml |
| 4.8 | 2.12 mg/ml |
| 4.1 | 2.03 mg/ml |

It can be seen that at a pH between 4 and 5, the solubility of the complexed delphinidin chloride increases by a factor of approximately 3 compared to a neutral pH.

### EXAMPLE 3

### Preparation of a Solid According to the Invention

In this example, a complex according to the invention is formulated as a solid. For comparative purposes, a delphinidin/HPBCD complex and a delphinidin/starch formulation are prepared in the form of a solid.

### EXAMPLE 3.1

### Delphinidin/SBE-β-CD

5 g of SEB-β-CD were dissolved in 40 ml of distilled water to give a clear solution. The pH of the solution was adjusted to 4.8 with 1 M HCl. 0.11 g of delphinidin chloride was then added and the mixture was stirred for 2 h at 27° C. in the dark. The homogeneous liquid was filtered under vacuum through a cellulose nitrate membrane filter of pore size 0.45 μm. The solution was frozen and subsequently freeze-dried at −48° C. and a pressure of approximately 10.3 Pa (77 mTorr). The lyophilizate was milled and sieved through a sieve of 0.3 mm mesh size.

### EXAMPLE 3.2

### Delphinidin/HPBCD

This was processed in the same manner as Example 3.1, but a significant amount of material was filtered off during the filtration which indicates that the solubilization was significantly less effective than using SBE-β-CD according to Example 3.1.

### EXAMPLE 3.3

### Delphinidin/starch formulation

5 g of starch was suspended in 40 ml of distilled water. A white suspension was obtained. The pH of the solution was adjusted to 4.6 with 1 M HCl. 0.11 g of delphinidin chloride was then added and the mixture was stirred at 27° C. for 2 h in the dark. The resulting homogeneous liquid was freeze-dried and the solid milled and sieved as in Example 3.1.

Example 3.1 is in accordance with the invention, while Examples 3.2 and 3.3 are comparative examples.

### EXAMPLE 4

### Stability Trials

The solids according to Examples 3.1 to 3.3 were stored under the following conditions:
8 days at room temperature in brown, screwtop glass containers,
then 22 days at room temperature in glass containers in the dark in an oxygen atmosphere.

The latter 22 days of the storage mentioned above were conducted in glass vials with a volume of 20 ml. In each case, 250 mg of the samples previously already stored for 8 days were placed therein, the vials were closed and sealed with a rubber stopper. By means of two injection needles, the head space of the vials was purged with pure oxygen. The samples were then stored in the dark.

The delphinidin content of the solids (calculated as delphinidin chloride and given in % by weight) was determined by the HPLC method described above. The results are given in Table 3 below.

| | Time elapsed (days) | | | | |
|---|---|---|---|---|---|
| | Start | 2 | 8 | 19 | 30 |
| Example 3.1 | 1.69 | 1.52 | 1.55 | 1.40 | 0.93 |
| Example 3.2 | 1.30 | 1.20 | 1.14 | 1.03 | 0.68 |
| Example 3.3 | 1.60 | 1.59 | 1.56 | 1.53 | 1.15 |

The results show that a delphinidin complex can be prepared in accordance with the invention which has good stability and thus good storage suitability even under a pure oxygen atmosphere. The complex also has good solubility in aqueous, particularly slightly acidic solutions, such that delphinidin may be formulated in accordance with the invention in a variety of ways. The stability of the solid according to the invention is just as good as a formulation with starch (Example 3.3), but this comparative example cannot be formulated as an aqueous solution.

### EXAMPLE 5

### Stability Trials in Aqueous Solution

To determine the delphinidin chloride content in the delphinidin-containing solutions, a reversed-phase HPLC method was used similar to the one already described above. The following reagents were used in this case:

Purified water

Methanol for chromatography

Formic acid, p.a.

1 M hydrochloric acid as a volumetric solution.

The column used was a Waters X Bridge™ C18, 35 μl, 150 mm×4.6 mm.

The mobile phases were as follows:
Phase A: Water 770 ml, methanol 230 ml, formic acid 10 ml
Phase B: Water 50 ml, methanol 950 ml, formic acid 10 ml

The following gradient program was used:

| Time [min] | Percent phase B |
|---|---|
| 0 | 0 |
| 5 | 0 |
| 20 | 20 |
| 25 | 100 |

Stop time: 25 min

After-run time (post time): 8 min

Flow rate: 1 ml/min

Injection volume: 20 μl

Column temperature: 30° C.+/−2° C.

UV/Vis detector: 530 μm for the assay, 275 μm for the detection of impurities

Integrator: area

Solutions and Sample Preparation:
Dilution solution 1: Mixture of 100 ml of methanol and 2.6 ml of 1 M HCl
Dilution solution 2: Mixture of 100 ml of 50% methanol and 2.6 ml of 1 M HCl

Calibration solution: A reference solution of delphinidin was prepared by weighing 10 mg of delphinidin chloride into a 10 ml flask and dissolving in dilution solution 1. After dissolution, the solution was diluted approximately 10-fold with dilution solution 2 to produce an approximate concentration of 0.1 mg/ml.

The control calibration solution was prepared in the same manner. The calibration solutions were immediately analyzed by HPLC since delphinidin chloride is unstable in solution.

Preparation of the Test Solutions:

To determine the delphinidin content of an aqueous solution according to the invention, delphinidin/SBE-β-CD from Example 3.1 (inventive) and delphinidin (comparative example) were dissolved in 0.9% NaCl solution until a starting concentration (based on the delphinidin) of 1.584 mg/ml (inventive example) or 0.0216 mg/ml (comparative example) had been established. The solutions were prepared at room temperature and subsequently stored in the dark at 37° C. in closed vials.

The delphinidin content was determined after 1, 2, 3 and 4 h. The table below gives the content determined as a percentage of the starting concentration stated above.

| | Non-complexed | Delphinidin/SBE- |
|---|---|---|
| Time [h] | delphinidin | β-CD |
| 0 | 100% | 100% |
| 1 | 8.3% | 80.7% |
| 2 | 6.5% | 74.5% |
| 3 | 5.6% | 64.7% |
| 4 | 5.1% | 62.8% |

The determination of the delphinidin content in the samples was calculated with the aid of the Agilent ChemStation software using the calibration with the external standard described.

II. Effect of the Anthocyanidin Delphinidin and the Delphinidin-SBE-β-CD Complex on Myeloma Cells In Vitro

1. Test Line and Experimental Setup
In the in vitro experiments, the effect of the delphinidin+sulfobutyl ether β-cyclodextrin complex (delphinidin+SBEBCD below) and delphinidin on the mouse myeloma cell line MOPC-315 (ATTC catalog no. TIB-23) was investigated by BLI (=bioluminescence imaging) measurement and FACS (fluorescence activated cell sorting) analysis described below. The methods used (BLI measurement and FACS analysis) are known to those skilled in the art from patent and technical literature, for example, from the FACS-based patent DE 1815352 C1.

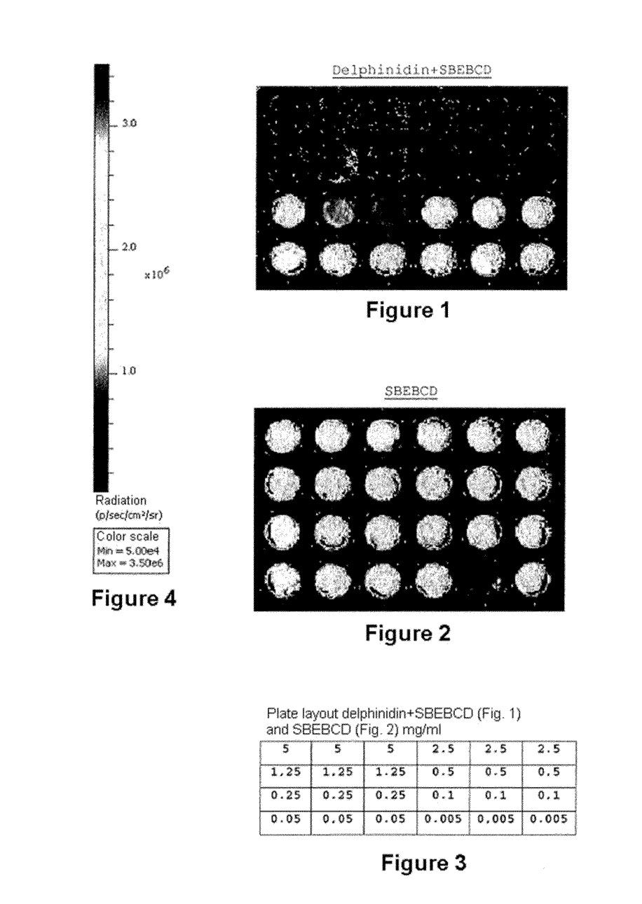

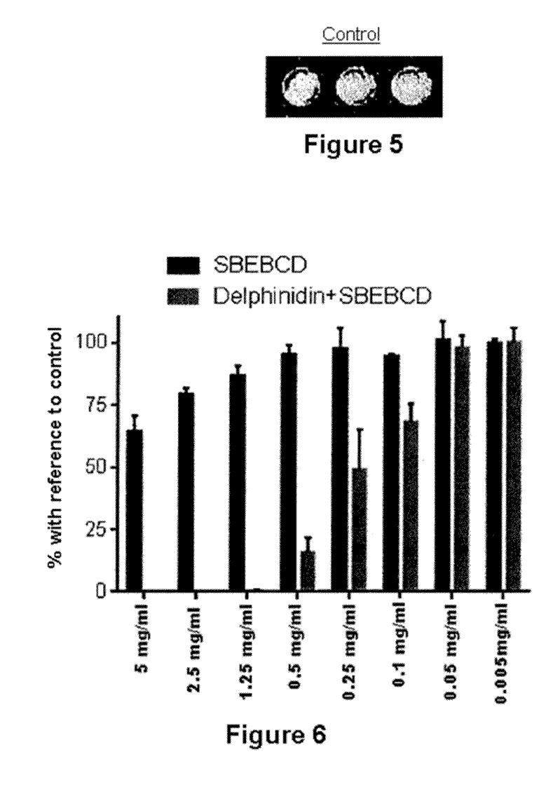

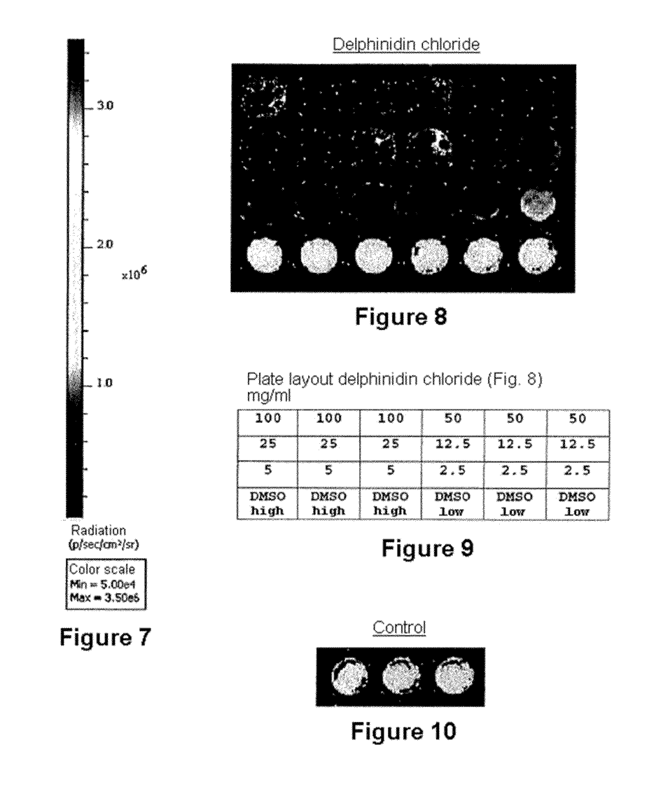

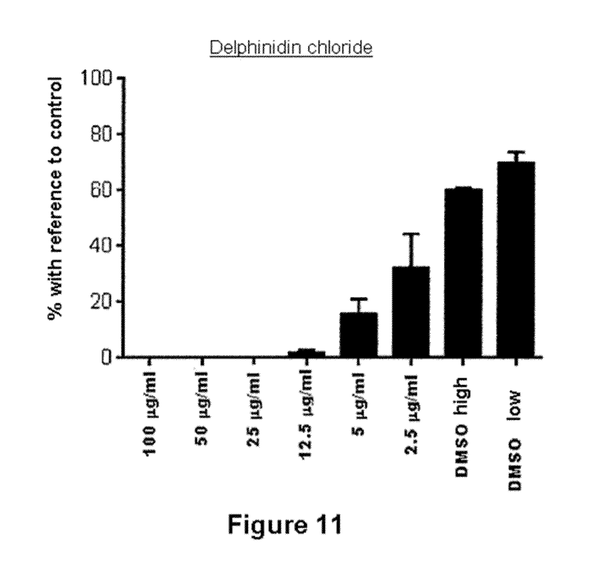

2. BLI Measurement
The results of the BLI measurement are presented in FIGS. 1 to 11 and give information on the number of cells surviving the treatment.
In an initial experiment, the effect of delphinidin+SBEBCD and SBEBCD was investigated. Firstly, cells in the exponential growth phase in 100 μl of RPMI-1640 cell medium were placed in a 24-well polystyrene cell culture plate (4000 cells/well). Sterile RPMI-1640 served as control. Delphinidin+SBEBCD or SBEBCD dissolved in 100 μl of RPMI-1640 were then added from a previously created dilution series as shown in FIG. 3 (all measurements in triplicate) and the cell culture plate was incubated at 37° C. for 48 hours, the medium subsequently exchanged for pure RPMI-1640 (i.e. fresh medium with no SBEBCD or delphinidin+SBEBCD) and the plate incubated again at 37° C. for 48 hours, for which the visual result is shown in FIGS. 1 (delphindin+SBEBCD), 2 (SBEBCD) and 5 (control).
The number of living cells in the well correlates with the number of emitted photons measured per well in the BLI measurement, which is expressed in the BLI FIGS. 1, 2 and 5 by corresponding colors (red=many signals/few emitted photons; blue=many signals/many emitted photons). FIG. 1 shows that with increasing dose strength of delphinidin+SBEBCD the toxicity increases up to the complete killing of all cells, while SBEBCD is barely toxic even in high doses, which is apparent from FIG. 2 (the well in the last row in FIG. 2 marked “X” has been excluded as an obvious test failure in view of the adjacent wells with the same concentrations). FIG. 6 summarizes the experimental results shown visually in FIGS. 1 (delphinidin+SBEBCD) and 2 (SBEBCD) again as a percentage based on the control (FIG. 5: medium only) as reference.
The effect of delphinidin as such was investigated using the same experimental setup. For this purpose, cells in the exponential growth phase in 100 μl of RPMI-1640 cell medium were likewise placed in a 24-well polystyrene cell culture plate (4000 cells/well). 100 μl of dissolved delphinidin chloride (dissolved in 10% DMSO and 90% H₂O) were then added in concentrations according to FIG. 9 (all measurements in triplicate) and the cell culture plate was incubated at 37° C. for 48 hours, the medium subsequently exchanged for pure RPMI-1640 (i.e. fresh medium with no delphinidin) and the plate incubated again at 37° C. for 48 hours, for which the visual result is shown in FIGS. 8 (delphindin chloride) and 10 (control: sterile RPMI-1640). In order to be able to check the effect of DMSO on the cells, two extra controls were added and also analyzed alongside [FIGS. 8 and 9, final row of wells in each case after addition of 100 μl of “DMSO high” (100 μg/ml DMSO) and 100 μl of “DMSO low” (50 μg/ml DMSO)]. FIG. 11 summarizes the experimental results shown visually in FIG. 8 (delphinidin chloride) again as a percentage based on the control (FIG. 10: medium only) as reference.

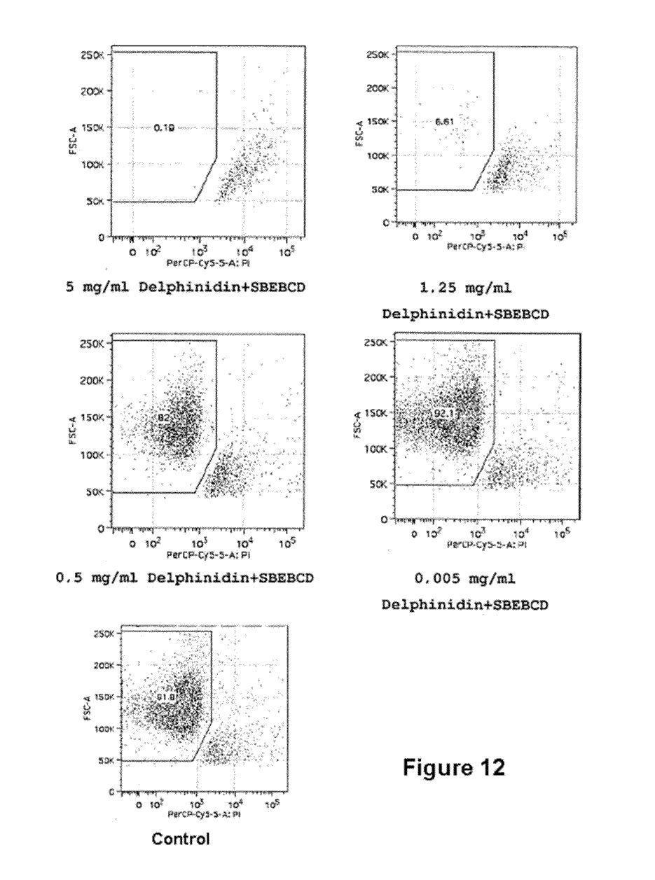

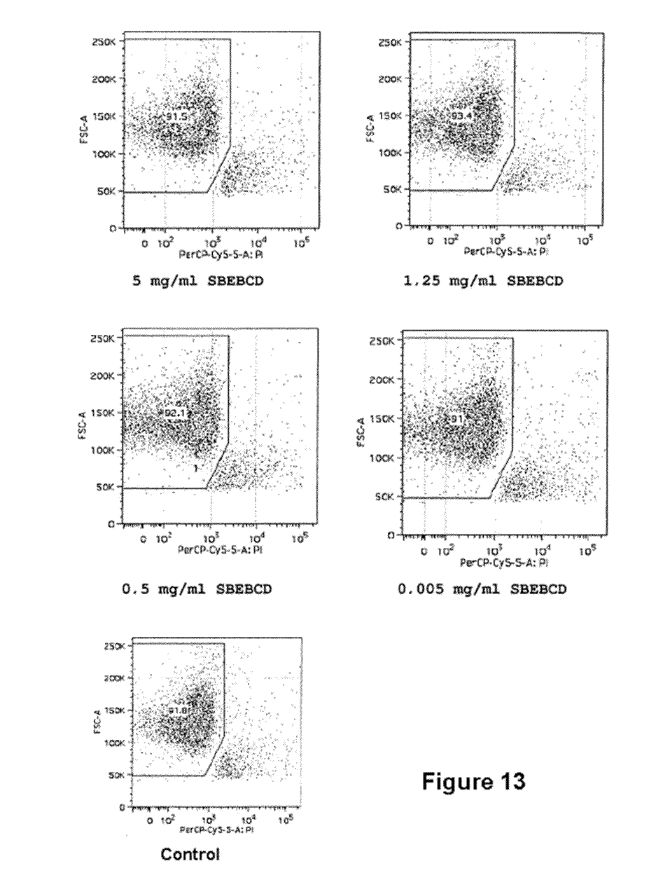

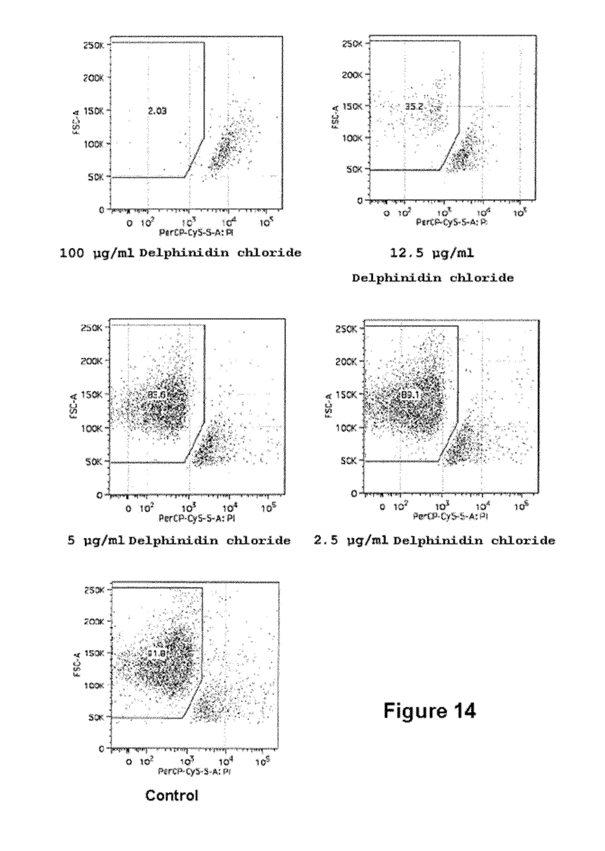

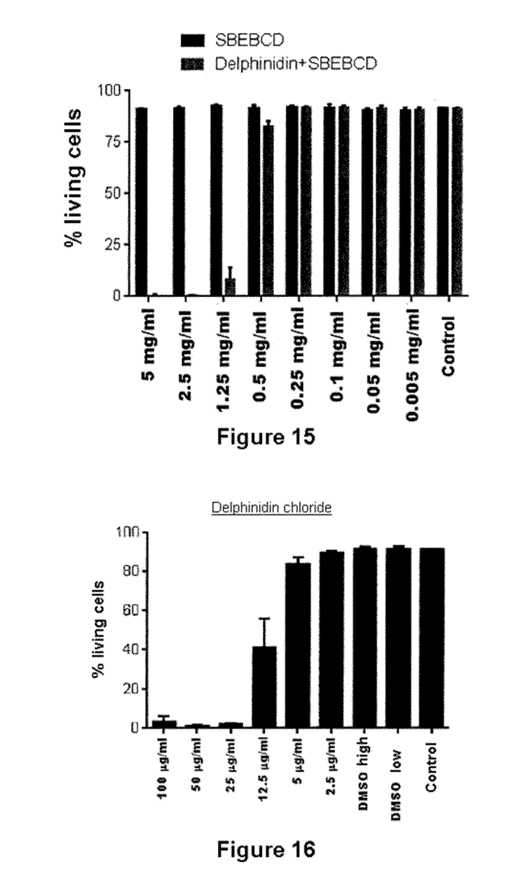

3. FACS Analysis
The results of the FACS analysis are shown in summary in FIGS. 12 (delphinidin+SBEBCD), 13 (SBEBCD), 14 (delphinidin chloride), 15 (delphinidin+SBEBCD and SBEBCD based on the control as reference) and 16 (delphinidin chloride, “DMSO high” and “DMSO low”, based in each case on the control as reference) and give information on the number of dead cells which had previously been stained with propidium iodide for the FACS analysis.
The experimental results from the BLI measurement and the FACS analysis can be summarized as follows:
   delphinidin+SBEBCD and delphinidin (delphinidin chloride) kill human myeloma cells in vitro.
   this effect increases in a dose-dependent manner wherein virtually all cells are killed at higher doses.

1. A complex of delphinidin and a sulfoalkyl ether β-cyclodextrin for use as a medicament.

2. The complex as claimed in claim 1 for use in the treatment of multiple myeloma.

3. The complex for use as claimed in either of the preceding claims, characterized in that the sulfoalkyl ether β-cyclodextrin is a sulfobutyl ether β-cyclodextrin.

4. The complex for use as claimed in any of the preceding claims, characterized in that the degree of substitution of the cyclodextrin with sulfoalkyl ether groups is 3 to 8, preferably 4 to 8, more preferably 5 to 8, more preferably 6 to 7.

5. The complex for use as claimed in any of the preceding claims 2-4, characterized in that the multiple myeloma is selected from the group consisting of stage I multiple myeloma, stage II multiple myeloma, stage III multiple myeloma, asymptomatic multiple myeloma, symptomatic myeloma, recently diagnosed multiple myeloma, responsive multiple myeloma, stable multiple myeloma, progressive multiple myeloma, relapsed multiple myeloma and refractive multiple myeloma.

6. The complex for use as claimed in any of the preceding claims, further comprising an effective amount of at least one therapeutic agent selected from the group consisting of bortezomib, melphalan, prednisone, vincristine, carmustine, cyclophosphamide, dexamethasone, thalidomide, doxorubicin, cisplatin, etoposide and cytarabine.

7. The complex for use as claimed in any of the preceding claims for use in the treatment of multiple myeloma in a subject undergoing or being prepared for radiation therapy and/or stem cell transplantation.

8. The complex for use as claimed in any of the preceding claims, further comprising a pharmaceutically acceptable carrier.

9. The complex for use as claimed in any of the preceding claims in a formulation form for administration in a form selected from the group consisting of oral, parenteral, including subcutaneous, intramuscular and intravenous, ophthalmic, pulmonary and nasal.

10. The complex for use as claimed in claim 9, characterized in that the oral administration form is a tablet or capsule.

11. The complex for use as claimed in claim 9, characterized in that the parenteral administration form is a solution, suspension or dispersion.

12. The complex for use as claimed in claim 9, characterized in that the ophthalmic, pulmonary or nasal administration form is an aerosol, solution, suspension or dispersion.
